(19) United States
(12) Patent Application Publication
(10) Pub. No.: US 2004/0253328 A1
(43) Pub. Date: Dec. 16, 2004

(54) Anti-atypical pneumonia decoction

(76) Inventors: Xin Yuan Hu, Shenzheng city (CN); XiMing Wang, Shenzheng city (CN); Jingshu Xue, Shenzheng city (CN)

Correspondence Address: JOHN S. PRATT, ESQ, KILPATRICK STOCKTON, LLP, 1100 PEACHTREE STREET, ATLANTA, GA, 30309, US

(21) Appl. No.: 10/624,577

(22) Filed: Jul. 21, 2003

(30) Foreign Application Priority Data
Jun. 13, 2003 (CN) 03137968.0

(51) Int. Cl. A61K 35/78

(52) U.S. Cl. 424/741; 424/757; 424/747; 424/763; 424/764; 424/773; 424/778

(57) ABSTRACT

An anti-atypical pneumonia decoction where a Chinese medicinal formula comprises the following medicinal materials: scutellaria root 0.5˜10 weight parts, gardenia 0.5˜10 weight parts, honeysuckle flower 0.5˜10 weight parts, weeping forsythia 0.5˜10 weight parts, dried rehmannia root 0.5˜10 weight parts, gypsum 0.5˜10 weight parts, flos chrysanthemi indici 0.5˜10 weight parts, basket fem 0.5˜10 weight parts, balloonflower root 0.5˜10 weight parts, mint 0.5˜10 weight parts, arctium fruit 0.5˜10 weight parts, and liquorice 0.5˜10 weight parts. Those medicinal materials were decocted with water to obtain the anti-atypical pneumonia decoction. The anti-atypical pneumonia decoction of the invention has satisfactory effect for preventing and treating atypical pneumonia or suspected atypical pneumonia.

### TECHNICAL FIELD

The present invention relates to a traditional Chinese medicine, especially a traditional Chinese medicinal formula, i.e., anti-atypical pneumonia decoction.

### BACKGROUND ART

Atypical pneumonia, which has fearful infectivity (also referred to as SARS, i.e., deep acute respiratory syndrome) is a disease caused by coronavirus. It spread over the world from 2002, especially in China. About ten thousand people have been infected with SARS, and quite a few of them died. Although a lot of human resource and funds have been used in many countries for research of the disease, no significant progress has obtained on the pathologic research so far. The pathogenesis is not known, and no vaccine and specific medicine have been developed. To date, the means for treating SARS according to Western medicine is to separate patient from public or treat patient depending on traditional experience only.

In this circumstance, against atypical pneumonia, the Western medicine based on anatomy and biochemistry loses its predominance compared with Chinese medicine that is mainly based on clinical experience. The advantage of Chinese medicine accumulated for thousand years cannot be instead by Western medicine in some instance. For example, to date in Beijing, more than one thousand SARS or suspect SARS patients have been treated with Chinese medicine or treated with combination of Chinese medicine and Western medicine. The number of people who are preventing from SARS by oral administration of Chinese medicine is too numerous to count. It is proved in practice that the combination of Chinese medicine and Western medicine has obtained satisfactory effect for preventing from atypical pneumonia or treating atypical pneumonia or suspect atypical pneumonia. This has been recognized by the World Health Organization (WHO) and all countries.

Although the incidence of SARS can be controlled in a period of time, it still takes a long time to die out. Moreover, in all probability, it might spread again as the change of climatic and other conditions change. Therefore, it is extremely necessary to provide a Chinese medicinal formula for preventing and treating atypical pneumonia or suspect atypical pneumonia with significant effect.

### DESCRIPTION OF THE INVENTION

In the view of above consideration, the problem solved by the present invention is to provide a Chinese medicinal formula for preventing and treating atypical pneumonia or suspect atypical pneumonia with significant effect, with an anti-atypical pneumonia decoction.

The present invention relates to an anti-atypical pneumonia decoction, characterized in that the Chinese medicinal formula consists of the following medicinal materials:

| Medicinal materials | Weight parts |
|---|---|
| Scutellaria root | 0.5˜10 |
| Gardenia | 0.5˜10 |
| Honeysuckle flower | 0.5˜10 |
| Weeping forsythia | 0.5˜10 |
| Dried rehmannia root | 0.5˜10 |
| Gypsum | 0.5˜10 |
| Flos chrysanthemi indici | 0.5˜10 |
| Basket fern | 0.5˜10 |
| Balloonflower root | 0.5˜10 |
| Mint | 0.5˜10 |
| Arctium fruit | 0.5˜10 |
| Liquorice | 0.5˜10 |

The medicinal materials above are decocted with water to obtain the anti-atypical pneumonia decoction.

According to the Chinese medicinal theory, atypical pneumonia and suspect atypical pneumonia belong to the epidemic disease of damp heat. Based on the experience of many years' treatment, the inventors have developed the said formula.

Aforementioned each of the medicinal materials has the following effects respectively. Scutellaria root with bitter cold has effect on heat-clearing and damp-drying. Gardenia with bitter cold has effect on toxic heat removing and cooling the blood and stopping bleeding. Honeysuckle flower with sweet in flavor and cold in nature has effect on toxic heat removing. Weeping forsythia with bitter and little cold has effect on toxic heat removing, treating boil and dispersing accumulation of pathogen. Dried rehmannia root has effect on cooling the blood and stopping bleeding. Gypsum with sweet in flavor and cold in nature has effect on heat-clearing, fire-purging, relieving restlessness and quenching thirst. Flos chrysanthemi indici with bitter cold has effect on toxic heat removing, removing heat from the blood and decreasing blood pressure. Basket fern with bitter, little cold and mild toxicity the effect on toxic heat removing and stopping bleeding. Balloonflower root with mild bitter and pungent has effect on resolving phlegm, arresting cough, evacuating pus and promoting the dispersing function of the lungs. Mint has the effect on dispelling wind and heat from the body and refreshing oneself with pungency in flavor and coolness in property. Arctium fruit with little cold has effect on relieving cough, reducing sputum and dispelling wind and heat from the body. Liquorice with mild sweet flavor has effect on lung nourishing, cough arresting and detoxification.

The formula of the invention is not conducted for special patients, but it is a formula which has confirmed and repeatable pharmacodynamics. There is not significant individual difference showed by the results of analysis of clinical pharmacodynamic data. It is proved in practice that the formula of the invention has significant effect for preventing and treating atypical pneumonia or suspect atypical pneumonia, due to the synergism of all medicinal materials above.

### BEST MODE FOR CARRYING OUT THE INVENTION

### EXAMPLE 1

According to the proportions showed as follows, the medicinal materials was mixed together to obtain the Chinese medicine formula:

| Medicinal materials | Weight (g) |
|---|---|
| Scutellaria root | 0.5 |
| Gardenia | 0.5 |
| Honeysuckle flower | 0.5 |
| Weeping forsythia | 0.5 |
| Dried rehmannia root | 0.5 |
| Gypsum | 0.5 |
| Flos chrysanthemi indici | 0.5 |
| Basket fern | 0.5 |
| Balloonflower root | 0.5 |
| Mint | 0.5 |
| Arctium fruit | 0.5 |
| Liquorice | 0.5 |

The said medicinal materials were decocted with water under slow fire for 40 minutes to obtain the anti-atypical pneumonia decoction. The anti-atypical pneumonia decoction obtained in this example is useful for children from two to four years old.

| Medicinal materials | Weight (g) |
|---|---|
| Scutellaria root | 10 |
| Gardenia | 10 |
| Honeysuckle flower | 10 |
| Weeping forsythia | 10 |
| Dried rehmannia root | 10 |
| Gypsum | 10 |
| Flos chrysanthemi indici | 10 |
| Basket fern | 10 |
| Balloonflower root | 10 |
| Mint | 10 |
| Arctium fruit | 10 |
| Liquorice | 10 |

The said medicinal materials were decocted with water using slow fire for 40 minutes to obtain the anti-atypical pneumonia decoction. The anti-atypical pneumonia decoction obtained in this example is useful for individuals whose age are above four years old.

Finally, the effect of the anti-atypical pneumonia decoction of the invention will be described further.

Prevention

36 volunteers were selected, in which 30 of them did not contact with any patient suffering from SARS, and 6 of them contacted with a patient suffering from SARS. Usage for the anti-atypical pneumonia decoction of example 1 or 2: recommended oral dose is 150 g per day administered as 75 g twice per day, 5 days, which is suitable for 2-4 year old children; recommended oral dose is 300 g per day administered as 150 g twice per day, 5 days, which is suitable for more than 4 year old.

As the result, no one of all 36 volunteers suffered from SARS.

Treatment

Criteria for choosing cases: 7 patients who were diagnosed as atypical pneumonia or suspect atypical pneumonia patients.

Criteria for diagnosis: diagnosed by means of Chinese medicine, patients have a continued fever, dry cough and upper dyspnea.

Regime of treatment: combination of Chinese medicine and Western medicine. Usage for the anti-atypical pneumonia decoction of example 1 or 2: recommended oral dose is 150 g per day administered as 75 g twice per day, which is suitable for 2-4 year old children; recommended oral dose is 300 g per day administered as 150 g twice per day, which is suitable for more than 4 year old.

Criteria for evaluation of therapeutic effectiveness: symptom elimination is considered as a standard of evaluation of therapeutic effectiveness.

Therapeutic effectiveness: after 3 doses of the anti-atypical pneumonia decoction of the invention administrated orally, 6 patients remove heat, stop coughing and become into eupnoea. It takes an average five days to recuperate their health completely from the day of initial administration.

Effect of prevention: effective rate is 100%.

Effect of treatment: 85% of patients have significant effect. For the 6 patients treated above, it takes an average five days to recuperate their health from the day of initial administration. For other patients that were not treated by the anti-atypical pneumonia decoction of the invention, it takes an average 29 days to recuperate their health; it takes an average 24 days less than that of patients treated by the anti-atypical pneumonia decoction of the invention.

All the facts indicate that the anti-atypical pneumonia decoction of the invention has significant effect for preventing and treating atypical pneumonia or suspect atypical pneumonia. It is a Chinese medicinal formula that possesses novelty, non-obviousness and practicability.

What is claimed:

1. Anti-atypical pneumonia decoction characterized by a Chinese medicinal formula comprising the following medicinal materials:

| Medicinal material | Weight parts |
|---|---|
| Scutellaria root | 0.5˜10 |
| Gardenia | 0.5˜10 |
| Honeysuckle flower | 0.5˜10 |
| Weeping forsythia | 0.5˜10 |
| Dried rehmannia root | 0.5˜10 |
| Gypsum | 0.5˜10 |
| Flos chrysanthemi indici | 0.5˜10 |
| Basket fern | 0.5˜10 |
| Balloonflower root | 0.5˜10 |
| Mint | 0.5˜10 |
| Arctium fruit | 0.5˜10 |
| Liquorice | 0.5˜10, |

Where, the said medicinal materials were decocted with water to obtain the anti-atypical pneumonia decoction.

2. Anti-atypical pneumonia decoction according to claim 1, characterized in that the Chinese medicinal formula comprises the following medicinal materials:

| Medicinal material | Weight parts |
|---|---|
| Scutellaria root | 0.5 |
| Gardenia | 0.5 |
| Honeysuckle flower | 0.5 |
| Weeping forsythia | 0.5 |
| Dried rehmannia root | 0.5 |
| Gypsum | 0.5 |
| Flos chrysanthemi indici | 0.5 |
| Basket fern | 0.5 |
| Balloonflower root | 0.5 |
| Mint | 0.5 |
| Arctium fruit | 0.5 |
| Liquorice | 0.5 |

3. Anti-atypical pneumonia decoction according to claim 1, characterized in that the Chinese medicinal formula comprises of the following medicinal materials:

| Medicinal material | Weight parts |
|---|---|
| Scutellaria root | 10 |
| Gardenia | 10 |
| Honeysuckle flower | 10 |
| Weeping forsythia | 10 |
| Dried rehmannia root | 10 |
| Gypsum | 10 |
| Flos chrysanthemi indici | 10 |
| Basket fern | 10 |
| Balloonflower root | 10 |
| Mint | 10 |
| Arctium fruit | 10 |
| Liquorice | 10 |
